(19) United States
(12) Patent Application Publication
(10) Pub. No.: US 2011/0076319 A1
(43) Pub. Date: Mar. 31, 2011

(54) BIORESORBABLE METAL STENT WITH CONTROLLED RESORPTION

(76) Inventors: Michael Orlowski, Bonn (DE); Alexander Rübben, Aachen (DE)

(21) Appl. No.: 12/524,702

(22) PCT Filed: Jan. 30, 2008
(86) PCT No.: PCT/DE2008/000161

§ 371 Date: May 7, 2010

Related U.S. Application Data
(60) Provisional application No. 60/899,636, filed on Feb. 6, 2007.

(30) Foreign Application Priority Data
Jan. 30, 2007 (DE) 10 2007 004 589.3

(51) Int. Cl. A61F 2/00; A61K 9/00; A61K 31/704; A61K 31/337; A61K 31/436; A61P 35/00; A61P 7/02; A61P 9/10

(52) U.S. Cl. 424/426; 424/400; 514/34; 514/449; 514/291

(57) ABSTRACT

Embodiments herein relate to a special form of a bioresorbable metal stent with controlled resorption due to a wrap with a special polymer, thereby ensuring a controlled resorption of the wrapped endoprosthesis subsequent to its implantation into a blood vessel. The resorbable implant comprises a magnesium alloy provided with a biodegradable coating. The biodegradable coating comprises biodegradable polymers and can further include at least one pharmacologically active substance such as an antiproliferative, antimigrative, antiangiogenic, antiinflammatory, antiphlogistic, cytostatic, cytotoxic and/or antithrombotic agent, anti-restenosis agents, corticoids, sex hormones, statins, epothilones, prostacyclins and/or inductors of angiogenesis.

### BACKGROUND OF THE INVENTION

1. Field of the Invention

The present invention relates to a special form of a bioresorbable metal stent (metallic endoprosthesis) with controlled resorption thanks to a wrap with a special polymer, thereby ensuring controlled resorption of the wrapped endoprosthesis subsequent to its implantation into a blood vessel. Therefore, the present invention relates to resorbable implants containing the metal magnesium and being provided with a biodegradable coating. The biodegradable coating includes biodegradable polymers and can further contain at least one pharmacologically active substance such as an antiproliferative, anti-migration, antiangiogenic, antiinflammatory, antiphlogistic, cytostatic, cytotoxic and/or antithrombotic agent, anti-restenosis agents, corticoids, sex hormones, statins, epothilones, prostacyclins and/or inductors of angiogenesis.

2. Description of the Relevant Art

Endoprostheses or stents supporting or keeping vessels open once they have been implanted into lesioned blood vessels, e.g. in the case of stenoses, dissections, etc. have been long-known in minimal-invasive interventional medicine. Usually, they are produced from metals such as stainless steel or nitinol. A large number of such metal stents is known and well established in practice. Due to their metal structure and carrying capacity, such metal stents are supposed to ensure that the vessels remain open after implantation and that the blood flow through the vessels is permanently guaranteed.

The supporting effect by the metal structure, however, is frequently only required for short periods of times as the body tissue can regenerate after the implantation of the stent.

For said reason, stents including bioresorbable materials, for example of polymers such as polylactide or of metals such as magnesium alloys have been developed in recent times and used in clinical trials.

While generally the aim to be achieved, namely resorption of the implanted stent, is achieved therewith, the problem that the degradation of the stent is not defined with respect to time is not to be neglected. Depending on the choice of material, design and material strength, degradation of the material varies strongly, cannot be controlled and generally happens too fast to ensure ingrowth of the stent into the vessel wall. In the case that the resorption happens too fast, the stent can not grow into the vessel wall. It is rather likely that it becomes detached and causes life-threatening complications in the patient.

For said reason, it is required to develop resorbable stents with controlled and defined degradation rate.

### SUMMARY OF THE INVENTION

Embodiments described herein relate to a prosthesis or vessel prosthesis which exercises its supporting function only until the regenerated tissue itself is once again capable of exercising that function and which can be biodegraded over a period of time in which the vessel reassumes its supporting function.

Said objective is resolved by the technical teaching of the independent claims. Further advantageous embodiments result from the dependent claims, the description and the examples.

One embodiment relates to a resorbable implant wherein the resorbable implant includes, to an extent of 40% by weight to 90% by weight of magnesium contained in a magnesium alloy and wherein the resorbable implant is covered with a biodegradable coating.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 demonstrates the degradation properties of four stents in aqua regia after about 9 minutes; and

FIG. 2 demonstrate the degradation properties of four stents in aqua regia after about 16 minutes.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

One embodiment relates to resorbable implants that include, predominantly, zinc, calcium an/or magnesium and have a biodegradable coating; furthermore, they may be capable of releasing corticoids, sex hormones, statins, epothilones, prostacyclins, inductors of angiogenesis or one or more antiproliferative, anti-migration, antiangiogenic, antiinflammatory, antiphlogistic, cytostatic, cytotoxic and/or antithrombotic agents or anti-restenosis agents.

The resorbable implant includes an amount of at least 40% by weight, preferably at least 50% by weight, more preferably at least 60% by weight, even more preferably at least 70% by weight, still more preferably at least 75% by weight, yet more preferably at least 80% by weight and most preferably at least 85% by weight of the metal magnesium, which is contained in a magnesium alloy with additional metals and possibly non-metals, metal salts, metal carbides, metal oxides and/or metal nitrides. Therefore, the amounts indicated in percent by weight refer to magnesium metal atoms and, if present, magnesium ions in the composition (alloy) with the other components.

Furthermore one embodiment of the implant has a content of calcium in the amount of 0-20% by weight, preferably 0.01-13% by weight, more preferably 0.1-8% per weight, yet more preferably 1-7% per weight. Most preferably, the amount of calcium is within the range of from 1.2-6.5% by weight, 1.4-6.0% by weight, 1.6-5.5% by weight, 1.8-5.0% by weight and especially from 2.0-4.5% by weight.

Furthermore one embodiment of the implant has a content of yttrium in the amount of 0-20% by weight, preferably 0.1-12% by weight, more preferably 0.5-6% per weight, yet more preferably 0.8-5% per weight. Most preferably, the amount of yttrium is within the range of from 0.9-4.0% by weight, 1.1-3.5% by weight, 1.3-3.0% by weight, 1.5-2.5% by weight and especially from 1.7-2.3% by weight.

In addition to zinc and optionally calcium and/or magnesium, an inventive implant may further contain at least one metal selected from the group comprising lithium, beryllium, sodium, aluminum, potassium, scandium, titanium, vanadium, chromium, manganese, iron, cobalt, nickel, copper, zinc, gallium, yttrium, zirconium, niobium, molybdenum, technetium, ruthenium, rhodium, palladium, silver, indium, tin, lanthanum, cerium, praseodymium, neodymium, promethium, samarium, europium, gadolinium, terbium, dysprosium, holmium, erbium, thulium, ytterbium, lutetium, tantalum, tungsten, rhenium, platinum, gold, lead and/or at least one metal salt with a cation selected from the group comprising Li⁺, Be²⁺, Na⁺, Mg²⁺, K⁺, Ca²⁺, Sc³⁺, Ti²⁺Ti⁴⁺, V²⁺, V³⁺, V⁴⁺, V⁵⁺, Cr²⁺, Cr³⁺, Cr⁴⁺, Cr⁶⁺, Mn²⁺, Mn³⁺, Mn⁴⁺, Mn⁵⁺, Mn⁶⁺, Mn⁷⁺Fe²⁺, Fe³⁺, Co²⁺, Co³⁺, Ni²⁺, Cu⁺, Cu²⁺, Zn²⁺, Ga⁺, Ga³⁺, Al³⁺, Y³⁺, Zr²⁺, Zr⁴⁺, Nb²⁺Nb⁴⁺, Nb⁵⁺, Mo⁴⁺, Mo⁶⁺, Tc²⁺, Tc³⁺, Tc⁴⁺, Tc⁵⁺, Tc⁶⁺, Tc⁷⁺, Ru³⁺, Ru⁴⁺, Ru⁵⁺, Ru⁶⁺, Ru⁷⁺, Ru⁸⁺, Rh³⁺, Rh⁴⁺, Pd²⁺, Pd³⁺, Ag⁺, In⁺, In³⁺, Ta⁴⁺, Ta⁵⁺, W⁴⁺, W⁶⁺, Pt²⁺, Pt³⁺, Pt⁴⁺, Pt⁵⁺, Pt⁶⁺, Au⁺, Au³⁺, Au⁵⁺, Sn²⁺, Sn⁴⁺, Pb²⁺, Pb⁴⁺, La³⁺, Ce³⁺, Ce⁴⁺, Gd³⁺, Nd³⁺, Pr³⁺Tb³⁺, Pr³⁺, Pm³⁺, Sm³⁺, Eu²⁺, Dy³⁺, Ho³⁺, Er³⁺, Tm³⁺, Yb³⁺. In addition to the aforementioned metals and metal salts which taken together are present in the amount of less than 10% by weight, preferably of less than 6% by weight and more preferably of less than 4% by weight, small amounts of non-metals, carbon, sulfur, nitrogen, oxygen and/or hydrogen may be present

The amounts of carbon (C), sulfur (S₈, Sₓ), nitrogen (N₂), oxygen (O₂) and/or hydrogen (H₂) including chemically bound carbon, sulfur, nitrogen, oxygen and/or hydrogen atoms, for example in form of metal carbides, metal oxides, metal nitrides together with the amount of the present rare earth metals and non-metals does not exceed the amount of 10% of the total composition. Rare earth metals, metal carbides, metal oxides, metal nitrides, non-metals, carbon, sulfur, nitrogen, oxygen, hydrogen are contained in the alloy in unavoidable traces of up to a maximum of 10% by weight, preferably in amounts of 0.1-8% by weight, more preferably 0.5-7.0% by weight, more preferably 1.0-6.0% by weight and most preferably 1.5-5.0% by weight.

One composition of an implant comprises for example

| | |
|---|---|
| 40% (w/w)-90%(w/w) | of magnesium |
| 0.0% (w/w)-20% (w/w) | of calcium |
| 0.0% (w/w)-20% (w/w) | of yttrium |
| 0.0% (w/w)-10% (w/w) | of other metals or metal salts |
| 0.0% (w/w)-10% (w/w) | of rare earth metals, metal carbides, |
| | metal oxides, metal nitrides, non- |
| | metals, carbon, sulfur, nitrogen, |
| | oxygen, hydrogen. |

The carbon, sulfur, nitrogen, oxygen, hydrogen or other non-metals or semi-metals may be present in form of anions and/or polymers.
Other compositions are as follows:

| | |
|---|---|
| 60% (w/w)-70% (w/w) | of magnesium |
| 10.0% (w/w)-20% (w/w) | of calcium |
| 5.0% (w/w)-15% (w/w) | of yttrium |
| 5.0% (w/w)-10% (w/w) | of other metals or metal salts |
| 2.0% (w/w)-10% (w/w) | of rare earth metals, metal carbides |
| | metal oxides, metal nitrides, non- |
| | metals, carbon, sulfur, nitrogen |
| | oxygen, hydrogen. |
| 70% (w/w)-80% (w/w) | of magnesium |
| 3.0% (w/w)-10% (w/w) | of calcium |
| 3.0% (w/w)-10% (w/w) | of yttrium |
| 1.0% (w/w)-7% (w/w) | of other metals or metal salts |
| 0.5% (w/w)-3% (w/w) | of rare earth metals, metal carbides |
| | metal oxides, metal nitrides, non- |
| | metals, carbon, sulfur, nitrogen |
| | oxygen, hydrogen. |
| 80% (w/w)-90% (w/w) | of magnesium |
| 1.0% (w/w)-5% (w/w) | of calcium |
| 1.0% (w/w)-3% (w/w) | of yttrium |
| 0.5% (w/w)-5% (w/w) | of other metals or metal salts |
| 0.3% (w/w)-1% (w/w) | of rare earth metals, metal carbides, |
| | metal oxides, metal nitrides, non- |
| | metals, carbon, sulfur, nitrogen, |
| | oxygen, hydrogen. |
| 80% (w/w)-90% (w/w) | of magnesium |
| 0.2% (w/w)-2% (w/w) | of calcium |
| 0.0% (w/w)-2% (w/w) | of yttrium |
| 8.0% (w/w)-10% (w/w) | of other metals or metal salts |
| 0.1% (w/w)-1% (w/w) | of rare earth metals, metal carbides, |
| | metal oxides, metal nitrides, non- |
| | metals, carbon, sulfur, nitrogen, |
| | oxygen, hydrogen. |
| 75% (w/w)-90% (w/w) | of magnesium |
| 0.1% (w/w)-5% (w/w) | of calcium |
| 0.5% (w/w)-3% (w/w) | of yttrium |
| 5.0% (w/w)-10% (w/w) | of zinc, iron, other metals or metal |
| | salts |
| 0.1% (w/w)-10% (w/w) | of rare earth metals, metal carbides, |
| | metal oxides, metal nitrides, non- |
| | metals, carbon, sulfur, nitrogen, |
| | oxygen, hydrogen. |

As used herein the term “resorbable” means that the implant slowly dissolves in the organism over a certain period of time until eventually only its degradation products in dissolved state are present in the body. At that moment, solid components or fragments of the implant are no longer present. The degradation products should largely be physiologically acceptable and should lead to ions or molecules which are present in the organism in any case or can be broken down into harmless substances or eliminated by the organism.

Metals which may be used in combination with the magnesium include the following: lithium, beryllium, sodium, zinc, aluminum, potassium, calcium, scandium, titanium, vanadium, chromium, manganese, iron, cobalt, nickel, copper, gallium, yttrium, zirconium, niobium, molybdenum, technetium, ruthenium, rhodium, palladium, silver, indium, tin, lanthanum, cerium, praseodymium, neodymium, promethium, samarium, europium, gadolinium, terbium, dysprosium, holmium, erbium, thulium, ytterbium, lutetium, tantalum, tungsten, rhenium, platinum, gold, lead. Zinc, calcium, iron, yttrium are particularly preferred. Further combinations include magnesium with or without the addition of one or more of the aforementioned metals with metal salts. Such combinations can be designated as zinc melts containing metal salts or as zinc alloys containing metal salts. The content of metal salts may only reach such a level at which a sufficient flexibility of the material remains guaranteed. In the case of stents, it is especially important that their capability to expand is not essentially affected. Suitable metal salts are those mentioned further below and especially salts from magnesium, zinc, calcium, iron and yttrium.

The use of resorbable alloys, however, is preferred with respect to the use of metals, wherein the metals may for example contain the following metals in combination with magnesium: lithium, beryllium, sodium, zinc, aluminum, potassium, calcium scandium, titanium, vanadium, chromium, manganese, iron, cobalt, nickel, copper, gallium, yttrium, zirconium, niobium, molybdenum, technetium, ruthenium, rhodium, palladium, silver, indium, tin, lanthanum, cerium, praseodymium, neodymium, promethium, samarium, europium, gadolinium, terbium, dysprosium, holmium, erbium, thulium, ytterbium, lutetium, tantalum, tungsten, rhenium, platinum, gold, lead. Sometimes such metals are only contained in small amounts.

Magnesium-zinc alloys containing zinc in an amount of 0.1 to 10% by weight, preferably 1.0 to 9.5% by weight and more preferably more than 4.0 to 9.0% by weight are preferred. Furthermore, in some embodiments, said magnesium-zinc alloy further contains scandium, titanium, vanadium, yttrium, zirconium, niobium, molybdenum, technetium, ruthenium, rhodium, palladium, silver or indium and especially yttrium in an amount of from 0.3-11%, preferably 0.7-10%, more preferably 1.1-8.5% and most preferred 2-7% by weight.

Alloys which in addition to magnesium predominantly contain calcium, zinc, iron, tin, zinc or lithium together with up to 10% by weight of scandium, yttrium, lanthanum, cerium, praseodymium, neodymium, promethium, samarium, europium, gadolinium, terbium, dysprosium, holmium, erbium, thulium and/or ytterbium are further preferred.

Furthermore, metal salts of the abovementioned metals may also be used. Such metal salts may include at least one of the following metal ions: Li⁺, Be²⁺, Na⁺, Mg²⁺, K⁺, Ca²⁺, Sc³⁺, Ti²⁺, Ti⁴⁺, V²⁺, V³⁺, V⁴⁺, V⁵⁺, Cr²⁺, Cr³⁺, Cr⁴⁺, Cr⁶⁺, Mn²⁺, Mn³⁺, Mn⁴⁺, Mn⁵⁺, Mn⁶⁺, Mn⁷⁺, Fe²⁺, Fe³⁺, Co²⁺, Co³⁺, Ni²⁺, Cu⁺, Cu²⁺, Zn²⁺, Ga⁺, Ga³⁺, Al³⁺, Y³⁺, Zr²⁺, Zr⁴⁺, Nb²⁺, Nb⁴⁺, Nb⁵⁺, Mo⁴⁺, Mo⁶⁺, Tc²⁺, Tc³⁺, Tc⁴⁺, Tc⁵⁺, Tc⁶⁺, Tc⁷⁺, Ru³⁺, Ru⁴⁺, Ru⁵⁺, Ru⁶⁺, Ru⁷⁺, Ru⁸⁺, Rh³⁺, Rh⁴⁺, Pd²⁺, Pd³⁺, Ag⁺, In³⁺, Ta⁴⁺, Ta⁵⁺, W⁴⁺, W⁶⁺, Pt²⁺, Pt³⁺, Pt⁴⁺, Pt⁵⁺, Pt⁶⁺, Au⁺, Au³⁺, Au⁵⁺, Sn²⁺, Sn⁴⁺, Pb²⁺, Pb⁴⁺, La³⁺, Ce³⁺, Ce⁴⁺, Gd³⁺, Nd³⁺, Pr³⁺, Tb³⁺, Pr³⁺, Pm³⁺, Sm³⁺, Eu²⁺, Dy³⁺, Ho³⁺, Er³⁺, Tm³⁺, Yb³⁺.

Anions used include halogens such as F⁻, Cl⁻, Br⁻, oxides and hydroxides such as OH⁻, O²⁻, sulfates, carbonates, oxalates, phosphates such as HSO₄⁻, SO₄²⁻, HCO₃⁻, CO₃²⁻, HC₂O₄⁻, C₂O₄²⁻, H₂PO₄⁻, HPO₄²⁻, PO₄³⁻, and especially carboxylates such as HCOO⁻, CH₃COO⁻, C₂H₅COO⁻, C₃H₇COO⁻, C₄H₉COO⁻, C₅H₁₁COO⁻, C₆H₁₃COO⁻, C₇H₁₅COO⁻, C₈H₁₇COO⁻, C₉H₁₉COO⁻, PhCOO⁻, PhCH₂COO⁻.

Furthermore, salts of the following acids may be used: sulfuric acid, sulfonic acid, phosphoric acid, nitric acid, nitrous acid, perchloric acid, hydrobromic acid, hydrochloric acid, formic acid, acetic acid, propionic acid, succinic acid, oxalic acid, gluconic acid, (glyconic acid, dextronic acid), lactic acid, malic acid, tartaric acid, tartronic acid (hydroxymalonic acid, hydroxypropanedioic acid), fumaric acid, citric acid, ascorbic acid, maleic acid, malonic acid, hydroxymaleic acid, pyruvic acid, phenylacetic acid, (o-, m-, p-) toluic acid, benzoic acid, p-aminobenzoic acid, p-hydroxybenzoic acid, salicylic acid, p-aminosalicylic acid, methanesulfonic acid, ethanesulfonic acid, hydroxyethanesulfonic acid, ethylenesulfonic acid, p-toluenesulfonic acid, naphthylsulfonic acid, naphthylaminesulfonic acid, sulfanilic acid, camphorsulfonic acid, china acid, quinic acid, o-methyl-mandelic acid, hydrogen-benzenesulfonic acid, methionine, tryptophan, lysine, arginine, picric acid (2,4,6-trinitrophenol), adipic acid, d-o-tolyltartaric acid, glutaric acid.

Furthermore, salts of amino acids containing for example one or more of the following amino acids may be used: glycine, alanine, valine, leucine, isoleucine, serine, threonine, phenylalanine, tyrosine, tryptophan, lysine, arginine, histidine, aspartate, glutamate, asparagine, glutamine, cysteine, methionine, proline, 4-hydroxyproline, N,N,N-trimethyllysine, 3-methylhistidine, 5-hydroxylysine, O-phosphserine, γ-carboxyglutamate, ε-N-acetyllysine, ω-N-methylarginine, citrulline, ornithine, Normally, amino acids having L-configuration are used. In another embodiment at least some of the amino acids used have D-configuration.

Other resorbable substances for the preparation of the implant are metal salts such as calcium chloride, calcium sulfate, calcium phosphate, calcium citrate, zinc chloride, zinc sulfate, zinc oxide, zinc citrate, iron sulfate, iron phosphate, iron chloride, iron oxide, zinc, magnesium chloride, magnesium sulfate, magnesium phosphate or magnesium citrate. Such metal salts are used in amounts of 0.01-10% by weight.

In another embodiment the magnesium alloy contains small amounts of a resorbable polymer. Said small amounts of a resorbable polymer are incorporated into the magnesium alloy, i.e. they are added to the alloy. Potentially suitable resorbable polymers are indicated below.

An expandable stent structure may be produced from the magnesium alloy. Said stent structure or said vessel prosthesis which is composed of the magnesium alloy is coated with a biodegradable polymer, i.e. each single strut of the stent or each single strut of the vessel prosthesis is provided with a biodegradable polymer layer, or respectively with a biodegradable polymer coating.

Polymer wraps or respectively coatings having a thickness of 0.2 μm and less are recommended.

The following polymers may be used as resorbable or biodegradable polymers:

polyvalerolactone, poly-ε-decalactone, polylactonic acid, polyglycolic acid, polylactides, polyglycolides, copolymers of the polylactides and polyglycolides, poly ε-caprolactone, polyhydroxybutyric acid, polyhydroxybutyrates, polyhydroxyvalerates, polyhydroxybutyrate-co-valerate, poly(1,4-dioxane-2,3-one), poly(1,3-dioxane-2-one), poly-para-dioxanone, polyanhydrides, polymaleic acid anhydride, polyhydroxy methacrylates, fibrin, polycyanoacrylate, polycaprolactone dimethylacrylates, poly-β-maleic acid, polycaprolactone butyl acrylates, multiblock polymers from oligocaprolactonediols and oligodioxanonediols, polyether ester multiblock polymers from PEG and poly(butylene terephthalates), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate), polyorthoesters, polyglycolic acid trimethyl carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide, soft polyurethanes, polyurethanes having amino acid residues in the backbone, polyetheresters such as polyethylene oxide, polyalkene oxalates, polyorthoesters as well as copolymers thereof, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivates thereof, heparins, chondroitin sulfate, dextran, β-cyclodextrins, copolymers with PEG and polypropylene glycol gum arabic, guar, gelatin, collagen, collagen N-hydroxysuccinimide, lipids, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylenes, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, polyiosbutylene, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones such as polysiloxanes, polydimethylsiloxanes, polyvinyl halogens and copolymers, cellulose ethers, cellulose triacetates, chitosans and copolymers and/or mixtures of the aforementioned polymers.

Resorbable polymers include polymethyl methacrylates (PMMA), polytetrafluoroethylene (PTFE), polyurethanes, polyvinyl chlorides (PVC), polydimethylsiloxanes (PDMS), polyesters, nylons and polylactides. Suitable polymer wraps further include especially parylenes, polyurethane or amino-ppx or semipermeable membranes.

In most cases, parylene is the term used for completely linear, partially crystalline, non-cross-linked aromatic polymers.

The different polymers have different properties and can be subdivided into four basic types, that is parylene C, parylene D, parylene N and parylene F, the structure of which is shown below:

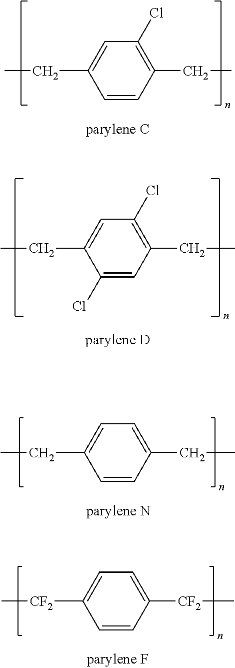

From the group of parylenes the monomer parylene N (poly-para-xylylene) has the simplest structure. Furthermore, there are the two chlorinated polymers parylene C (chloropoly-para-xylylene) and parylene D (di-chloro-poly-para-xylylene). In the case of parylene F (poly(tetrafluoro-para-xylylene)) the methyl units are fluorinated.

With only 290° C., parylene C has the lowest melting point of the aforementioned parylenes and is characterized by good mechanical properties and corrosion resistance to corrosive gases as well as very low permeability to moisture. Parylene C is a biocompatible polymer and can thus be used in physiological environments.

Polyesters, polylactides as well as copolymers of diols and esters, or respectively diols and lactides may also be used. Diols used include, for example, ethane-1,2-diol, propane-1,3-diol or butane-1,4-diol.

Polyesters may be used for the polymer layer. From the group of polyesters, such polymers having the following repeat unit may be used:

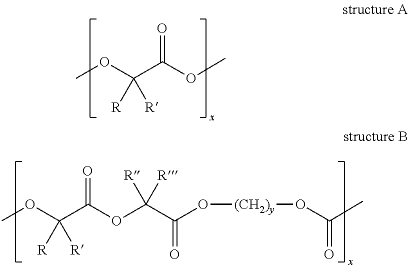

In the repeat units shown R, R′, R″ and R′″ represent an alkyl residue having from 1 to 5 carbon atoms, in particular methyl, ethyl, propyl, isopropyl, n-butyl, s-butyl, t-butyl, iso-butyl, n-pentyl or cyclopentyl. In some embodiments, the alkyl residue is methyl or ethyl. Y represents an integer from 1 to 9 and X represents the degree of polymerization. The following polymers including the repeat units shown may be used:

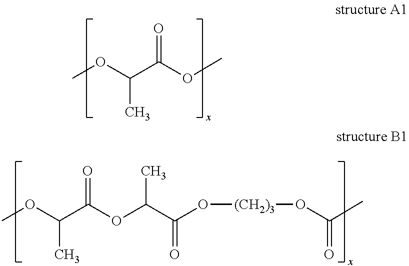

Specific polymers that may be used include the polymers Resomer® R 203 and Resomer® LT 706.

The name Resomer® represents a highly technological product of the company Boehringer Ingelheim, which product, as a medical device made from resorbable polymers, is an important alternative to conventional medical applications, due to the progresses that have been achieved with respect to technical developments and the fact that it is applicable in various fields.

Said resorbable polymers are produced on the basis of lactic acid and glycolic acid. Homopolymers from lactic acid (polylactides) are mainly used for the production of resorbable, medical implants. Copolymers of lactic acid and glycolic acid are used as raw materials in the preparation of capsules containing active agents for controlled release of pharmaceutical active substances.

Thus, polymers on the basis of lactic acid and glycolic acid as well as copolymers (alternating or statistic copolymers) and block copolymers (e.g. triblock copolymers) of both acids may be used.

Other representatives of the resorbable polymers Resomer® include poly(L-lactide)s of the general formula —(C₆H₈O₄)ₙ— such as L 210, L 210 S, L 207 S, L 209 S, poly(L-lactide-co-D,L-lactide)s of the general formula —(C₆H₈O₄)ₙ— such as LR 706, LR 708, L 214 S, LR 704, poly(L-lactide-co-trimethyl carbonate)s of the general formula —[(C₆H₈O₄)ₓ—(C₄H₆O₃)_{y}]ₙ— such as LT 706, poly(L-lactide-co-glycolide)s of the general formula —[(C₆H₈O₄)ₓ—(C₄H₄O₄)_{y}]ₙ— such as LG 824, LG 857, poly(L-lactide-co-ε-caprolactone)s of the general formula —[(C₆H₈O₄)ₓ—(C₆H₁₀O₂)_{y}]ₙ— such as LC 703, poly(D,L-lactide-co-glycolide)s of the general formula —[(C₆H₈O₄)ₓ—(C₄H₄O₄)_{y}]ₙ— such as RG 509 S, RG 502H, RG 503H, RG 504H, RG 502, RG 503, RG 504, poly(D,L-lactide)s of the general formula —(C₆H₈O₄)ₙ— such as R 202 S, R 202H, R 203 S and R 203H. Resomer® 203 S is the successor of the polymer Resomer®R 203. In some embodiments R 203 and LT 706 are used in a ratio of 70% by weight to 30% by weight.

One embodiment concerns implants such as stents provided with a biodegradable coating of a polymethyl methacrylate (PMMA), polytetrafluoroethylene (PTFE), polyurethane, polyvinyl chloride (PVC), polydimethylsiloxane (PDMS), polyester, nylon or polylactide and particularly a polyester and/or polylactide. The metallic inner part of said implants is composed as disclosed herein, namely of magnesium and optionally calcium or yttrium, including the additional components mentioned further above.

The inventive resorbable implants have an inner structure including the magnesium alloy wrapped by the biodegradable polymer, wherein the coating or the wrap is only provided around the single struts and the stent structure is not completely coated or wrapped, i.e. the interspaces between the single struts are not covered by the biodegradable polymer.

In an embodiment a controlled resorption rate is achieved by wrapping a resorbable stent with an insoluble or hardly soluble polymer coating and by providing the polymer wrap, once it has been applied onto the resorbable stent, with holes ensuring a resorption of the stent situated within said wrap.

For example, such holes can be produced mechanically, chemically or optically by lasering. In lieu of the perforated, insoluble or only hardly soluble polymer wrap, it is also possible that a continuous, semipermeable polymer wrap is applied onto the stent.

A degradation and removal of said wrap by macrophages in the blood upon resorption of the stent can be ensured by choosing suitable polymers and a suitable thickness of said polymers.

In another embodiment, the metallic stent is provided with a coating allowing for a fluid permeation.

In one embodiment, a bioresorbable magnesium stent is provided with a coating that includes a polyurethane, a parylene or amino-ppx or a mixture of the aforementioned substances, wherein the coating is provided with holes which are created mechanically or chemically or optically by laser treatment once the coating has been applied onto the metal stent.

Furthermore, in an embodiment a medical implant, includes more than 70% by weight, preferably to more than 80% by weight, yet more preferably to more than 85% by weight and most preferably to more than 90% by weight of the magnesium alloy, and the biodegradable polymer coating is provided in an amount corresponding to the remaining weight percentage.

Additional embodiments include resorbable implants that include at least one pharmacologically active substance on the implant and/or in the implant or underneath the resorbable or respectively biodegradable layer and/or in the biodegradable layer and/or on the biodegradable layer. Pharmacologically active substances include antiproliferative, anti-migration, antiangiogenic, antiinflammatory, antiphlogistic, cytostatic, cytotoxic and/or antithrombotic agents, anti-restenosis agents, corticoids, sex hormones, statins, epothilones, prostacyclins, inductors of angiogenesis. In some embodiments the antiproliferative, antiinflammatory, antiphlogistic, cytostatic, cytotoxic and/or antithrombotic agents and the anti-restenosis agents are used.

Examples for antiproliferative, anti-migration, antiangiogenic, antiinflammatory, antiphlogistic, cytostatic, cytotoxic antithrombotic agents and/or anti-restenosis agents include: abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a, 4-hydroxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, inhibitor 2ω of smc proliferation, mycophenolate mofetil, c-myc antisense, b-myc antisense, β-lapachone, podophyllotoxin, podophyllic acid 2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, NFkB, angiopeptine, monoclonal antibodies inhibiting muscle cell proliferation, bFGF antagonists, probucol, prostaglandins, 1-hydroxy-11-methoxycanthin-6-one, scopoletin, NO donors such as pentaerythritol tetranitrate and sydnonimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof such as 6-α-hydroxy-paclitaxel, taxoteres, carbon suboxide (MCS) and macrocylic oligomers thereof, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF 1, active agents from the group of antibiotics such as cefadroxil, cefazolin, cefaclor, cefoxitin, tobramycin, gentamicin, penicillins such as dicloxacillin, oxacillin, sulfonamides, metronidazole, enoxaparin, desulfated and N-reacetylated heparin, tissue plasminogen activator, GpIIb/IIIa platelet membrane receptor, antibodies to factor Xa inhibitor, heparin, hirudin, r-hirudin, PPACK, protamine, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators such as p65, NF-kB or Bcl-xL antisense oligonucleotides, halofuginone, nifedipine, tocopherol, molsidomine, tea polyphenols, epicatechin gallate, epigallocatechin gallate, leflunomide, etanercept, sulfasalazine, tetracycline, triamcinolone, mutamycin, procainimide, retinoic acid, quinidine, disopyrimide, flecainide, propafenone, sotalol, natural and synthetically obtained steroids such as inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, clotrimazole, flucytosine, griseofulvin, ketoconazole, miconazole, nystatin, terbinafine, antiprotozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid baccharinoids B1, B2, B3 and B7, tubeimoside, bruceantinoside C, yadanziosides N and P, isodeoxyelephantopin, tomenphantopin A and B, coronarin A,B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, marchantin A, maytansin, lycoridicin, margetine, pancratistatin, liriodenine, oxoushinsunine, periplocoside A, deoxypsorospermin, psychorubin, ricin A, sanguinarine, manwu wheat acid, methylsorbifolin, chromones of spathelia, stizophyllin, mansonine, strebloside, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

In some embodiments paclitaxel and derivatives thereof such as 6-α-hydroxy-paclitaxel or baccatin or other taxoteres, sirolimus, tacrolimus, everolimus, Gleevec (imatinib), erythromycin, midecamycin, josamycin and triazolopyrimidine may be used.

Paclitaxel (Taxol®) as well as all derivatives of paclitaxel, such as 6-α-hydroxy-paclitaxel are used in some embodiments.

The inventive resorbable implants may act as supporting prostheses for channel-like structures and particularly vessel prostheses and stents for blood vessels, urinary tracts, respiratory tracts, biliary tracts or the digestive tract.

Among said stents, stents for blood vessels or more generally for the circulatory system, i.e. for the cardiovascular area, are used in some embodiments

Generally the stents concerned are auto-expandable or can be expanded by means of a balloon, wherein said stents may include an antiproliferative, anti-migration, antiangiogenic, antiinflammatory, antiphlogistic, cytostatic, cytotoxic, anti-restenotic, antithrombotic and/or an anti-restenosis agent.

Generally, the biodegradable polymer layer serves as a carrier for the at least one active agent, such as the antiproliferative, anti-migration, antiangiogenic, antiinflammatory, antiphlogistic, cytostatic, cytotoxic, antithrombotic agent and/or anti-restenosis agent. Thanks to said agent, inflammations which could be induced by the stent are prevented and especially the growth of smooth muscle cells (coronary endothelial cells) on the stent is regulated. The stent allows for a regeneration of the supported tissue or of the supported tissue section. Once the tissue has regenerated, it is capable of supporting the vessel autonomously without further support by the stent being required. At that moment, the stent which will have grown into the vessel wall will already be degradated to a significant degree. The degradation processes continue until the stent is completely degradated, but without said stent being broken down into solid fragments which would be able to move freely in the bloodstream.

The terms “resorbable” or “degradable” or “biodegradable” refer to the fact that the human or animal body is capable of slowly decomposing the implant into components which are present in dissolved state in the blood or in other body fluids.

Stents may have a grid-like structure, wherein the single bars of the grid structure have similar cross sectional areas. A ratio of less than 2 between the largest and the smallest cross-sectional area may be used. The similar cross-sectional areas of the bars lead to a steady degradation of the stent.

Furthermore the ring-shaped bars may be connected by connecting bars, wherein said connecting bars preferably have a smaller cross-sectional area or a smaller minimum diameter than the bars forming the ring-shaped bars. Thus, the connecting bars are degradated more quickly in the human or animal body than the ring-shaped bars. By said degradation of the connecting bars, the axial flexibility of the stent increases more rapidly than the carrying capacity of the stent decreases as a result of the degradations of the ring-shaped bars.

The medical implant, in particular the stent, can be coated by using a spraying or dipping method, wherein a polymer is dissolved in a solvent and said solution is applied onto the implant.

Suitable solvents include water and organic solvents such as chloroform, methylene chloride (dichloromethane), acetone, tetrahydrofuran (THF), diethyl ether, methanol, ethanol, propanol, isopropanol, diethyl ketone, dimethylformamide (DMF), dimethylacetamide, acetic acid ethyl ester, dimethyl sulfoxide (DMSO), benzene, toluene, xylene, t-butyl methyl ether (MTBE), petroleum ether (PE), cyclohexane, pentane, hexane, heptane. In some embodiments a suitable solvent is chloroform or methylene chloride.

The at least one active agent to be applied can be dissolved, emulated, suspended or dispersed in a suitable solvent or even together with the polymer.

Potential substances to be applied include the pharmacologically active agents mentioned above and the polymers described above.

### EXAMPLES

### Example 1

A stent includes:

| | |
|---|---|
| 89% w/w | of magnesium |
| 7% w/w | of calcium |
| 1% w/w | of zinc |
| 2% w/w | of yttrium |
| 1% w/w | of other metals, metal salts, non-metals, rare earth metals, |
| | metal carbides, metal oxides, metal nitrides, carbon, sulfur, |
| | nitrogen, oxygen, hydrogen. |

The stent according to example 1 is coated in a dipping process with a solution of a polyglycol and doxorubicin. Upon drying, the dipping process is repeated two more times.

### Example 2

A stent includes:

| | |
|---|---|
| 81% w/w | of magnesium |
| 11% w/w | of calcium |
| 2% w/w | of zinc |
| 3% w/w | of yttrium |
| 3% w/w | of other metals, metal salts, non-metals, rare earth metals, |
| | metal carbides, metal oxides, metal nitrides, carbon, sulfur, |
| | nitrogen, oxygen, hydrogen. |

The stent according to example 2 is coated in a spraying process with a solution of a polylactide and the active agent paclitaxel in dimethyl sulfoxide or in methanol. The spraying process is repeated several times.

### Example 3

A stent includes:

| | |
|---|---|
| 72.0% w/w | of magnesium |
| 7.3% w/w | of calcium |
| 3.2% w/w | of yttrium |
| 2.1% w/w | of manganese |
| 2.8% w/w | of iron |
| 4.8% w/w | of zinc |
| 7.8% w/w | of other metals, metal salts, non-metals, rare earth metals, |
| | metal carbides, metal oxides, metal nitrides, carbon, sulfur, |
| | nitrogen, oxygen, hydrogen. |

The stent according to example 3 is coated in a spraying process with a solution of a polyester containing the active agent rapamycin in chloroform. The spraying process is repeated several times.

### Example 4

A stent includes:

| | |
|---|---|
| 85.0% w/w | of magnesium |
| 4.6% w/w | of calcium |
| 2.9% w/w | of yttrium |
| 0.7% w/w | of manganese |
| 3.8% w/w | of iron |
| 2.5% w/w | of zinc |
| 0.5% w/w | of other metals, metal salts, non-metals, rare earth metals, |
| | metal carbides, metal oxides, metal nitrides, carbon, sulfur, |
| | nitrogen, oxygen, hydrogen. |

The stent according to example 4 is coated in a spraying process with a solution of a polyamide in acetone without any pharmacological active agent being included. The spraying process is repeated several times.

### Example 5

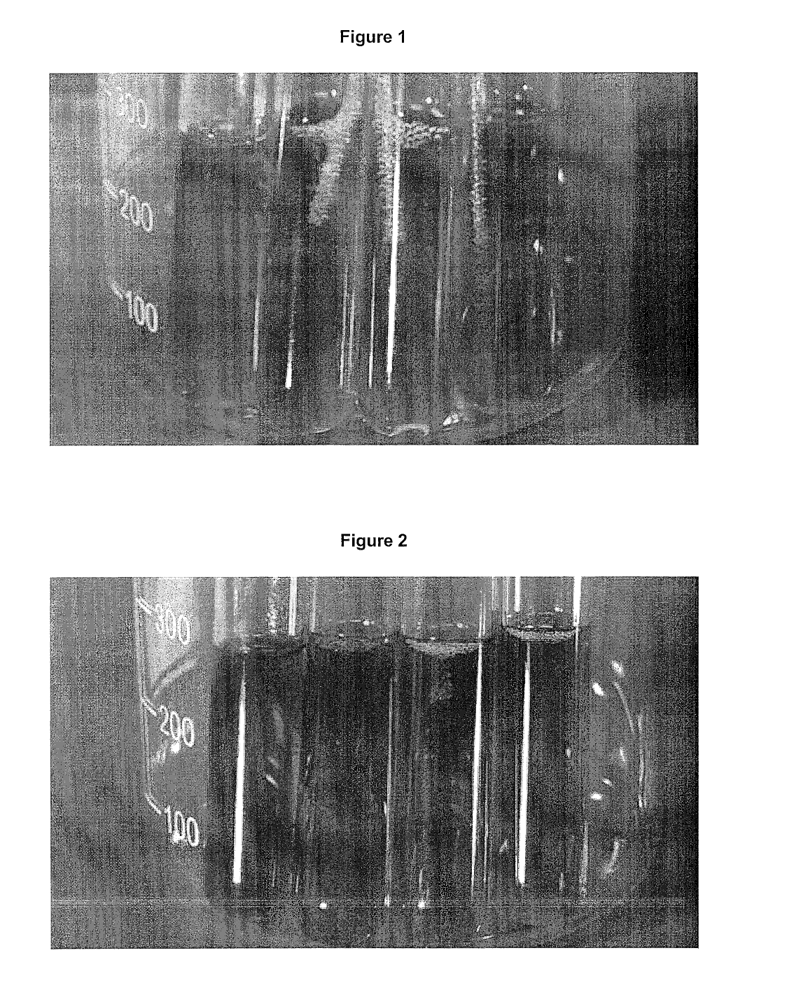

Three uncoated stents composed according to example 4 (length 27 mm, mass 26.5 mg) are coated as follows:
Stent number 1: uncoated
Stent number 2: CVD-coated with polyamino-p-xylylen-co-poly-p-xylylen (short amino-ppx), coating with a high number of pores (total area of pores makes about 25% of the surface of the stent)
Stent number 3: CVD-coated with amino-ppx, coating with a low number of pores (total area of pores makes about 10% of the surface of the stent)
Stent number 4: CVD coated with amino-ppx, unmodified standard coating according to the work instruction CVD-coatings
   Afterwards the degradation properties in aqua regia (hydrochloric acid (concentrated)/nitric acid (concentrated) 3:1 (v/v)) were tested. There four stents were put simultaneously into four test-tubes filled with aqua regia. The course of the degradation of the stent was observed visually and documented by video tape recordings and photography. The time that passed until the stents were completely degraded was recorded. It is referred to FIGS. 1 and 2.

The times which passed till each of the stents was completely degraded are compared in the following:

Stent: time until complete oxidative degradation:
Stent number 1 about 7 minutes
Stent number 2 about 12 minutes
Stent number 3 about 15 minutes
Stent number 4 about 30 minutes
   It is obvious that the CVD-coating increases the resistance of the metal towards the oxidative reagent considerably. The standard coating (stent number 4) even results in a four times increased “life time” of the stent. Furthermore the experiment demonstrates that the progression of the oxidative disengagement or dissolution of the stent can be regulated by varying the porosity of the coating.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. Resorbable implant, wherein the resorbable implant comprises to an extent of 40% by weight to 90% by weight of magnesium contained in a magnesium alloy and wherein the resorbable implant is covered with a biodegradable coating.

2. Resorbable implant according to claim 1, wherein the resorbable implant further contains up to 10% by weight of calcium.

3. Resorbable implant according to claim 1, wherein the resorbable implant further contains up to 10% by weight of yttrium.

4. Resorbable implant according to claim 1, wherein the magnesium alloy of the resorbable implant is composed as follows:

| | |
|---|---|
| 40% (w/w)-90% (w/w) | of magnesium |
| 0.0% (w/w)-20% (w/w) | of calcium |
| 0.0% (w/w)-20% (w/w) | of yttrium |
| 0.0% (w/w)-10% (w/w) | of other metals or metal salts |
| 0.0% (w/w)-10% (w/w) | of rare earth metals, metal carbides, |
| | metal oxides, metal nitrides, non-metals, |
| | carbon, sulfur, nitrogen, oxygen, hydrogen. |

5. Resorbable implant according to claim 1, wherein the resorbable implant further contains at least one metal selected from the group comprising lithium, beryllium, sodium, magnesium, aluminum, potassium, calcium, scandium, titanium, vanadium, chromium, manganese, iron, cobalt, nickel, copper, zinc, gallium, yttrium, zirconium, niobium, molybdenum, technetium, ruthenium, rhodium, palladium, silver, indium, tin, lanthanum, cerium, praseodymium, neodymium, promethium, samarium, europium, gadolinium, terbium, dysprosium, holmium, erbium, thulium, ytterbium, lutetium, tantalum, tungsten, rhenium, platinum, gold, lead.

6. Resorbable implant according to claim 1, wherein the metal salt comprises at least one metal ion in the following oxidation states: Li⁺, Be²⁺, Na⁺, Mg²⁺, K⁺, Ca²⁺, Sc³⁺, Ti²⁺, Ti⁴⁺, V²⁺, V³⁺, V⁴⁺, V⁵⁺, Cr²⁺, Cr³⁺, Cr⁴⁺, Cr⁶⁺, Mn²⁺, Mn³⁺, Mn⁴⁺, Mn⁵⁺, Mn⁶⁺, Mn⁷⁺, Fe²⁺, Fe³⁺, Co²⁺, Co³⁺, Ni²⁺, Cu⁺, Cu²⁺, Zn²⁺, Ga⁺, Ga³⁺, Al³⁺, Y³⁺, Zr²⁺, Zr⁴⁺, Nb²⁺, Nb⁴⁺, Nb⁵⁺Mo⁴⁺Mo⁶⁺, Tc²⁺, Tc³⁺, Tc⁴⁺, Tc⁵⁺, Tc⁶⁺, Tc⁷⁺, Ru³⁺, Ru⁴⁺, Ru⁶⁺, Ru⁷⁺, Ru⁸⁺, Rh³⁺, Rh⁴⁺, Pd²⁺, Pd³⁺, Ag⁺, In⁺, In³⁺, Ta⁴⁺, Ta⁵⁺, W⁴⁺, W⁶⁺, Pt²⁺, Pt³⁺, Pt⁴⁺, Pt⁵⁺, Pt⁶⁺, Au⁺, Au³⁺, Au⁵⁺, Sn²⁺, Sn⁴⁺, Pb²⁺, Pb⁴⁺, La³⁺, Ce³⁺, Ce⁴⁺, Gd³⁺, Nd³⁺, Pr³⁺, Tb³⁺, Pr³⁺, Pm³⁺, Sm³⁺, Eu²⁺, Dy³⁺, Ho³⁺, Er³⁺, Tm³⁺, Yb³⁺.

7. Resorbable implant according to claim 1, wherein the biodegradable coating is a polymer coating comprises natural, synthetic and semisynthetic polymers.

8. Resorbable implant according to claim 7, wherein the biodegradable coating comprises at least one of the following biodegradable substances or of mixtures of the following biodegradable substances: polyvalerolactone, poly-ε-decalactone, polylactonic acid, polyglycolic acid, polylactides, polyglycolides, copolymers of the polylactides and polyglycolides, poly-ε-caprolactone, polyhydroxybutyric acid, polyhydroxybutyrates, polyhydroxyvalerates, polyhydroxybutyrate-co-valerate, poly(1,4-dioxane-2,3-one), poly(1,3-dioxane-2-one), poly-para-dioxanone, polyanhydrides, polymaleic acid anhydride, polyhydroxy methacrylates, fibrin, polycyanoacrylate, polycaprolactone dimethylacrylates, poly-β-maleic acid, polycaprolactone butyl acrylates, multiblock polymers from oligocaprolactonediols and oligodioxanonediols, polyether ester multiblock polymers from PEG and poly(butylene terephthalate), polypivotolactones, polyglycolic acid trimethyl carbonates, polycaprolactone glycolides, poly(□-ethyl glutamate), poly(DTH-iminocarbonate), poly(DTE-co-DT-carbonate), poly(bisphenol A-iminocarbonate), polyorthoesters, polyglycolic acid trimethyl carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide, soft polyurethanes, polyurethanes having amino acid residues in the backbone, polyetheresters such as polyethylene oxide, polyalkene oxalates, polyorthoesters as well as copolymers thereof, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivatives thereof, heparins, chondroitin sulfate, dextran, β-cyclodextrins, copolymers with PEG and polypropylene glycol, gum arabic, guar, gelatin, collagen N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylenes, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones such as polysiloxanes, polydimethylsiloxanes, polyvinyl halogens and copolymers, cellulose ethers, cellulose triacetates, chitosans and copolymers and/or mixtures of the aforementioned polymers.

9. Resorbable implant according to claim 8, wherein the biodegradable coating comprises a polyester, polyurethane, parylene, amino-ppx, polylactide or a mixture of two or more of the aforementioned polymers.

10. Resorbable implant according to claim 1, wherein the coating allows for a fluid permeation.

11. Resorbable implant according to claim 1, wherein the biodegradable coating is a semipermeable membrane.

12. Resorbable implant according to claim 1, wherein the coating is provided with holes.

13. Resorbable implant according to claim 12, wherein, subsequent to the coating, the holes in the coating are created mechanically, chemically or optically by lasering.

14. Resorbable implant according to claim 1, wherein at least one pharmacologically active substance is provided on and/or in the resorbable implant.

15. Resorbable implant according to claim 1, wherein at least one pharmacologically active substance is provided on, in and/or underneath the biodegradable coating.

16. Resorbable implant according to claim 14, wherein the at least one pharmacologically active substance is selected from the group comprising antiproliferative, antimigrative, antiangiogenic, antiinflammatory, antiphlogistic, cytostatic, cytotoxic and/or antithrombotic agents, anti-restenosis agents, corticoids, sex hormones, statins, epothilones, prostacyclins, inductors of angiogenesis.

17. Resorbable implant according to claim 16, wherein the at least one pharmacologically active substance is selected from the group comprising: abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a, 4-hydroxyoxy cyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, inhibitor-2□ of smc proliferation, mycophenolate mofetil, c-myc antisense, b-myc antisense, β-lapachone, podophyllotoxin, podophyllic acid 2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, NFkB, angiopeptin, monoclonal antibodies inhibiting muscle cell proliferation, bFGF antagonists, probucol, prostaglandins, 1-hydroxy-11-methoxycanthin-6-one, scopoletin, NO donors such as pentaerythritol tetranitrate and sydnonimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof such as 6-α-hydroxy-paclitaxel, taxoteres, carbon suboxide (MCS) and macrocylic oligomers thereof, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF 1, active agents from the group of antibiotics such as cefadroxil, cefazolin, cefaclor, cefoxitin, tobramycin, gentamicin, penicillins such as dicloxacillin, oxacillin, sulfonamides, metronidazole, enoxaparin, desulfated and N-reacetylated heparin, tissue plasminogen activator, GpIIb/IIIa platelet membrane receptor, antibodies to factor Xa inhibitor, heparin, hirudin, r-hirudin, PPACK, protamine, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators such as p65, NF-kB or Bcl-xL antisense oligonucleotides, halofuginone, nifedipine, tocopherol, molsidomine, tea polyphenols, epicatechin gallate, epigallocatechin gallate, leflunomide, etanercept, sulfasalazine, tetracycline, triamcinolone, mutamycin, procainimide, retinoic acid, quinidine, disopyrimide, flecamide, propafenone, sotalol, natural and synthetically obtained steroids such as inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, clotrimazole, flucytosine, griseofulvin, ketoconazole, miconazole, nystatin, terbinafine, antiprotozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceantinoside C, yadanziosides N and P, isodeoxyelephantopin, tomenphantopin A and B, coronarin A, B, C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, marchantin A, maytansin, lycoridicin, margetine, pancratistatin, liriodenine, oxoushinsunine, periplocoside A, deoxypsorospermin, psychorubin, ricin A, sanguinarine, manwu wheat acid, methylsorbifolin, chromones of spathelia, stizophyllin, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

18. Resorbable implant according to claim 1, wherein the resorbable implant is a stent for blood vessels, urinary tracts, respiratory tracts, biliary tracts or the digestive tract.
